(19) United States
(12) Patent Application Publication
(10) Pub. No.: US 2002/0055184 A1
(43) Pub. Date: May 9, 2002

(54) SYSTEMS FOR DETECTING ANALYTES

(76) Inventors: STEPHEN NAYLOR, ROCHESTER, MN (US); JOHN F. O'BRIEN, ORONOCO, MN (US); H. ROBERT BERGEN III, ROCHESTER, MN (US)

Correspondence Address: MARK S ELLINGER, FISH & RICHARDSON PC, 60 SOUTH SIXTH STREET, SUITE 3300, MINNEAPOLIS, MN, 55402

(21) Appl. No.: 09/391,432

(22) Filed: Sep. 8, 1999

(51) Int. Cl. G01N 33/558

(52) U.S. Cl. 436/514

(57) ABSTRACT

Systems for detecting analytes that include an immunoaffinity cartridge, a preconcentrator cartridge, and a mass spectrometer are described. The systems also can include a membrane cartridge. Methods for detecting the presence or absence of an analyte in a biological sample are described.

### TECHNICAL FIELD

The invention relates to systems for detecting analytes that include an affinity cartridge, a preconcentrator cartridge, and a mass spectrometer.

### BACKGROUND OF THE INVENTION

Detection of carbohydrate deficient isoforms of transferrin (CDT) has been used to diagnose carbohydrate deficient glycoprotein syndrome (CDGS) and chronic alcohol consumption. See, Stibler et al., Alcohol Clin. Exp. Res., 1981, 5:545-549; and Stibler et al., Arch. Dis. Child, 1990, 65:107-111. CDGS is a genetic syndrome that is most often clinically apparent in infancy or childhood due to retardation of mental and motor skills. Other gross morphological features occur and insufficiencies of some glycosylated peptide hormones also are demonstrable. In CDGS, transferrin isoforms are shifted from penta and tetra sialylated forms to di- and asialyl forms. The glycoprotein abnormalities are caused by a number of synthetic defects in N-glycosylation. The most frequent such defect is under-production of mannose due to phosphomannose mutase deficiency. Mannose is the branch point hexose in most N-glycosylated proteins.

The originally described method of resolving CDTs from the more abundant transferrin species was isoelectric focusing (IEF). Five or more sialic acid residues contribute to transferrin's acidic isoelectric point (pI) and loss of this functionality results in a basic shift within the pI range of 5.2 to 5.9. Current methods of CDT detection use combinations of IEF followed by Western blotting for specific identification, or immunopurification/IEF followed by Coomassie staining of transferrin isoforms. Resolution of isotransferrins for CDGS diagnosis is done by IEF and has been combined with preliminary one-step immunoaffinity purification. More recently, ion exchange chromatography followed by immunoassay of transferrin in eluates has been used for isolating low and high sialylated forms of transferrin. High performance liquid chromatography (HPLC) of transferrin isoforms also has been accomplished. Such methods resolve transferrins based on charge and indicate the presence or absence of terminal sialic acids, but are not sensitive to internal structural absences of neutral monosaccharide moieties. Methods that employ IEF, considered the gold standard, are technically difficult for routine laboratory use. Ion exchange/ immunoquantitation methods also are somewhat cumbersome, but, more importantly, are subject to error of inclusion of higher sialylated forms in the CDT fraction unless pH and ionic strength of the eluent are strictly maintained.

The sensitivity attained with IEF easily detects CDT at the levels found in serum derived from CDGS patients. Sensitivity and specificity of assaying CDT for chronic alcoholism is subject to variation depending on patient selection. The continued evaluation of CDT as a marker for alcoholism suggests that its presence, though undoubtedly due to alcoholism, is more subtle than might be gleaned from some of the previous investigations. This may be due to performance of current methods used for quantitation of CDT. Given the gravity of the implications for labeling persons as being alcohol dependant, more sensitive methods for detecting CDT levels are needed.

### SUMMARY OF THE INVENTION

The invention is based on the discovery that systems that include an affinity cartridge, a preconcentrator cartridge, and a mass spectrometer are useful for detecting analytes such as transferrin, from a biological sample. The invention allows analytes to be rapidly detected with minimal fluid handling steps. Detailed information about the structure of the analytes, such as changes in oligosaccharide content between protein isoforms, also is provided by the invention. Furthermore, multiple analytes can be detected, allowing a profile of analytes to be examined in a single biological sample.

The invention features a system for detecting an analyte that includes an immunoaffinity cartridge, a preconcentrator cartridge, and a mass spectrometer, wherein the preconcentrator cartridge operably connects the immunoaffinity cartridge and the mass spectrometer. The preconcentrator cartridge includes a container having an inner surface, an outer surface, a first port, a second port, and a membrane disposed inside the container in contact with the inner surface of the container such that a liquid sample that enters the container through the first port and exits the container through the second port traverses the membrane, wherein the membrane includes a chemically inert organic polymer matrix embedded with absorbent particles. The immunoaffinity cartridge can include an anti-transferrin antibody that is polyclonal or monoclonal. The mass spectrometer can be an electrospray ionization-mass spectrometer such as a microspray electrospray ionization-mass spectrometer or a nanospray electrospray ionization-mass spectrometer.

The system further can include a membrane cartridge, wherein the membrane cartridge is operably connected to the immunoaffinity cartridge.

The invention also features a system for detecting an analyte that includes a membrane cartridge, an immunoaffinity cartridge, a preconcentrator cartridge, and a mass spectrometer, wherein the membrane cartridge is operably connected to the immunoaffinity cartridge, the immunoaffinity cartridge is operably connected to the preconcentrator cartridge, and the preconcentrator cartridge is operably connected to the mass spectrometer.

The membrane cartridge includes a membrane, such as a chemically inert organic polymer matrix of a defined pore size. The membrane cartridge can include a container having an inner surface, an outer surface, a first port, a second port, and a membrane disposed inside the container in contact with the inner surface of the container such that a liquid sample that enters the container through the first port and exits the container through the second port traverses the membrane.

The preconcentrator cartridge includes a membrane such as a chemically inert organic polymer matrix embedded with absorbent particles. The preconcentrator cartridge can include a container having an inner surface, an outer surface, a first port, a second port, and a membrane disposed inside the container in contact with the inner surface of the container such that a liquid sample that enters the container through the first port and exits the container through the second port traverses the membrane.

The mass spectrometer can be an electrospray ionization-mass spectrometer such as a microspray electrospray ionization-mass spectrometer or a nanospray electrospray ionization-mass spectrometer.

The immunoaffinity cartridge can include an anti-transferrin antibody that is polyclonal or monoclonal.

In another aspect, the invention features a method for detecting the presence or absence of an analyte in a biological sample. The method includes eluting biological macromolecules from a membrane cartridge comprising a membrane, wherein the membrane includes the biological sample such as a blood sample; immunopurifying the analyte from the eluted biological macromolecules; concentrating the immunopurified analyte; and detecting the presence or absence of the concentrated analyte by mass spectrometry, wherein the eluting, immunopurifying, concentrating, and detecting steps are performed on-line. The biological macromolecules and the analyte can each comprise polypeptides. The analyte can be, for example, carbohydrate-deficient transferrin.

The membrane cartridge can include a container having an inner surface, an outer surface, a first port, a second port, and the membrane disposed inside the container in contact with the inner surface of the container such that a liquid sample that enters the container through the first port and exits the container through the second port traverses the membrane. The membrane can be a chemically inert organic polymer matrix of a defined pore size and further can include an internal standard. The internal standard can be transferrin lacking sialic acid residues.

A method of evaluating a patient for the presence or absence of an analyte in a biological sample from the patient is also featured. The method includes providing a sample collection kit to the patient, wherein the kit includes a membrane and instructions for obtaining the biological sample and applying the biological sample to the membrane to form a sample membrane; receiving the sample membrane from the patient; inserting the sample membrane into a membrane cartridge; eluting biological macromolecules from the membrane cartridge; immunopurifying the analyte from the eluted biological macromolecules; concentrating the immunopurified analyte; and detecting the presence or absence of the concentrated analyte by mass spectrometry, wherein the eluting, immunopurifying, concentrating, and detecting steps are performed on-line.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a schematic representation of a system for detecting analytes.

FIG. 2 is a schematic representation of a preconcentrator cartridge.

FIG. 3 is a schematic representation of a membrane and a membrane cartridge.

FIG. 4 is a schematic representation of a system for detecting multiple analytes from a biological sample.

FIG. 5 is an IEF gel of neuraminidase digested transferrin (lanes labeled 0, 30 min., and 24 hrs. reflect time of exposure to neuraminidase), samples from CDGS patients (lanes 1-3) and a chronic alcoholic (lane 4), and a control sample (lane 5).

FIG. 6A and 6B are mass spectra of neuraminidase treated samples for 0 hrs. (6A) and 24 hrs. (6B).

FIGS. 7A-7B are mass spectra of a sample obtained from a control patient (7A) and a sample from a CDGS patients (7B).

FIG. 8 is a mass spectrum of a sample obtained from a patient suspected of chronic alcohol abuse.

FIG. 9 is a mass spectrum of a blood sample from an alcoholic spiked with a purified internal standard.

### DETAILED DESCRIPTION

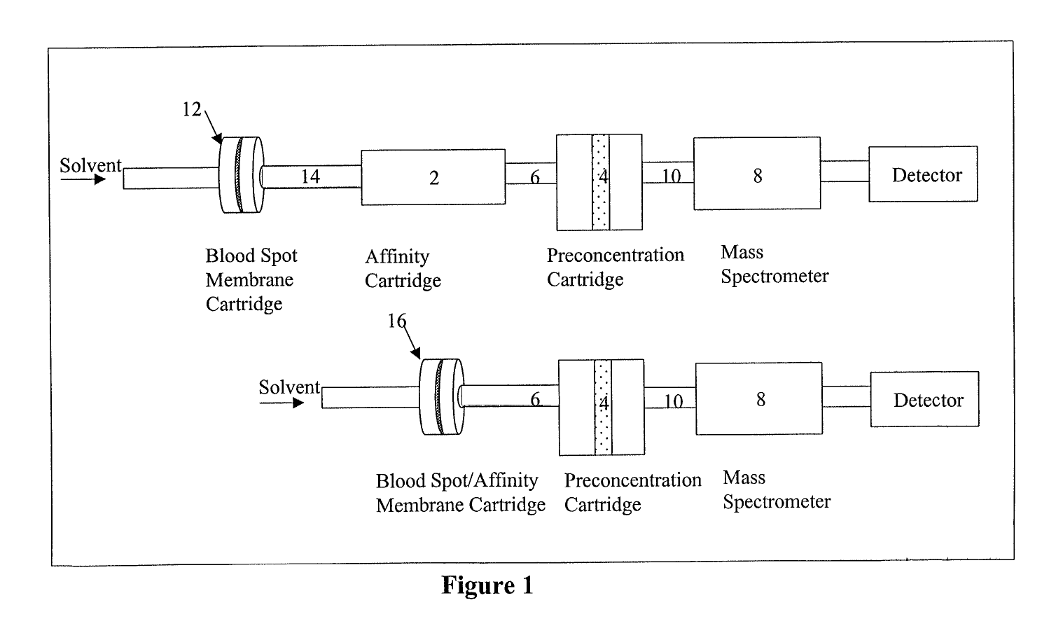

The invention features systems for detecting analytes. As used herein, the term “analyte” refers to a biological macromolecule to be detected in a sample, such as a nucleic acid, a lipid, or a polypeptide. Polypeptide refers to a chain of amino acids of any length, regardless of post-translational modifications such as glycosylation. For example, the analyte can be a blood polypeptide such as transferrin or ceruloplasmin. As depicted in FIG. 1, the system broadly includes affinity cartridge 2 operably connected to preconcentration cartridge 4 via fluid connectors such as tubing 6, and preconcentration cartridge 4 operably connected to mass spectrometer 8 via fluid connectors such as tubing 10. The system further can include membrane cartridge 12 operably connected to affinity cartridge 2 via tubing 14. In some embodiments, affinity membrane cartridge 16 is used in place of affinity cartridge 2. As used herein, “operably connected” refers to attachment of the system components in a manner such that each component can function on-line with the other components. Tubing 6, 10, or 14 preferably is chemically non-reactive (i.e., inert) and is not degraded or otherwise affected by buffers, reagents, or analytes used in systems of the invention. Tubing 6, 10, or 14 can be composed of, for example, an organic polymer such as polytetrafluorethylene (PTFE), polyetheretherketone (PEEK), or polyethylene, or stainless steel. Fluid connectors typically have a diameter of about 1 μm to about 400 μm.

Each component in the system is configured with appropriate hardware for operably connecting via fluid connectors to another component. In addition, each component can be configured with hardware for controlling solvent flow. Thus, each component in the system may contain, for example, clamps, valves, or joints on inlet and/or outlet ports.

Biological samples can be introduced into the inlet of membrane cartridge 12 or affinity cartridges 2 or 16, or spotted directly onto the membrane of membrane cartridge 16. The inlet of membrane cartridge 12, affinity cartridge 2, or affinity membrane cartridge 16 can be fitted with a standard injection mechanism.

### Affinity Cartridges

Affinity cartridges contain a solid phase derivatized with an agent that adsorbs, adheres, or otherwise binds, covalently or noncovalently, the analyte of interest. Affinity cartridges can have a single compartment or can have multiple compartments. An example of a multiple-compartment affinity cartridge is a multi-well plate (e.g., 96 wells) adapted for fluid flow across the plate with a frit or other suitable means. The affinity cartridge can be an immunoaffinity cartridge, which includes a solid phase derivatized with an antibody having specific binding affinity for the analyte of interest. Antibodies can be polyclonal or monoclonal. For example, an immunoaffinity cartridge used in a system for detecting carbohydrate deficient isoforms of transferrin (CDT) would contain an antibody having specific binding affinity for transferrin.

The solid phase can be a membrane, as in affinity membrane cartridge 16, or other solid support, as in affinity cartridge 2. Suitable membranes for derivatizing with affinity agents such as antibodies are composed of a chemically inert organic polymer matrix, such as PTFE or TEFLON™.

An example of another solid support that can be used to prepare an affinity cartridge is POROS A Self Pack Media (PerSeptive BioSystems, Framingham, Mass.). For example, rabbit anti-human transferrin polyclonal antibody (Dako Corp., Carpinteria, Calif.) can be covalently linked to POROS A medium or other suitable support medium per manufacturer's instructions, and unreacted sites can be blocked with a buffer such as Tris, to produce an affinity medium. Affinity medium can be packed into a small column, e.g. 0.1 cm×2 cm, to generate an affinity cartridge of the invention. Affinity medium is retained in the small column through a porous material which allows liquid to pass while retaining the affinity medium in the column. Materials such as wool, silica, a polymer, ceramic, or metal can be used to construct one or more frits or plugs, which retain the affinity medium, but allow solvent to flow through. Alternatively, the affinity medium is retained by narrowing, constricting, or crimping the internal wall or surface of the column to confine the affinity medium, while allowing liquids to pass. The column typically is composed of material to which analytes do not adhere, including glass or metal.

The solid phase also can be derivatized with, for example, lectins such as Concanavalin A or wheat gern lectin, which have affinity for glycoproteins and proteoglycans; heparin, which has affinity for many proteins including lipoproteins and proteases; boronate, which adsorbs cis-hydroxyls, such as those found in glycoproteins, an organomercurial crosslinked matrix, which has affinity for sulfhydryl proteins; or a dye such as blue dextran or cibacron Blue (Sigma Chemical Company, St. Louis, Mo.).

### Preconcentrator Cartridge

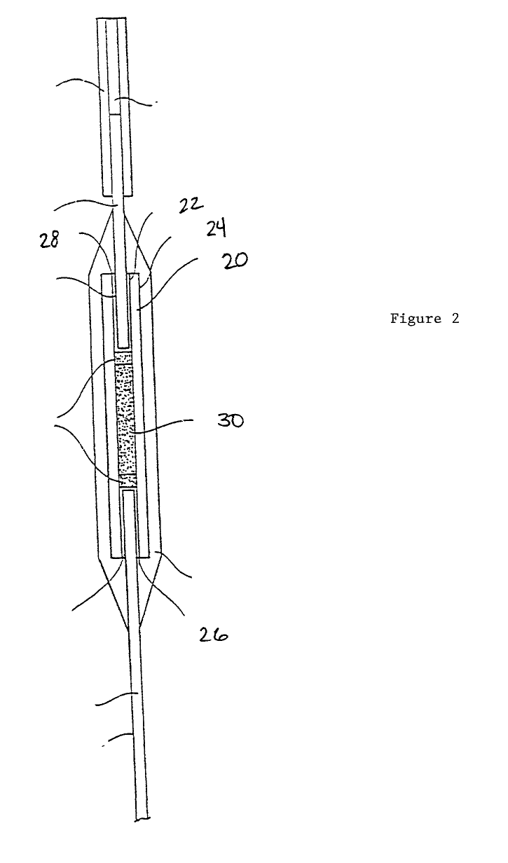

Preconcentration cartridges concentrate the analyte of interest up to about 10⁴ fold, and allow flow rate to be reduced to nanoliters per minute, as required by microspray or nanospray mass spectrometers. In one embodiment, as depicted in FIG. 2, preconcentrator cartridge 4 includes a container 20 having an inner surface 22, an outer surface 24, a first port 26, a second port 28, and a membrane 30 disposed inside the container in contact with inner surface 22 of container 20. A liquid sample that enters container 20 through first port 26 and exits container 20 through second port 28 traverses membrane 30. See, U.S. Pat. No. 5,800,692. Suitable membranes are composed of a chemically inert organic polymer matrix, such as PTFE, or, for example, Teflon™, embedded with absorbent particles, such as silica. Membranes can be derivatized with an aliphatic hydrocarbyl group such as C2, C8, or C18 alkyl groups or poly(styrene divinylbenzene). For example, EMPORE™ membranes (3M Company, St. Paul, Minn.), which are Teflon™ lattice membranes impregnated with solid phase beads, are particularly useful. Membranes typically are less than about 1 mm thick.

Container 20 can be fabricated from a material to which analytes do not adhere, such as a metal, metalloid, glass, ceramic, graphite, organic polymer, or a composite of such materials (e.g. graphite-spiked polymer), and which is able to withstand high pressure. Fluorinated hydrocarbon polymers such as polyfluorotetraethylene (Teflon™) are particularly useful in fabricating the container. The container can be of any shape, including, for example, cylindrical, spherical, or cubical.

Alternatively, preconcentrator cartridges can be solid phase absorbing pre-columns composed, for example, of reversed phase silica such as C18, C8, C4, C2, and poly(styrene divinylbenzene) phenyl, phenyl ether, and ethyl ether media. Suitable pre-columns are available commercially from Phenomenex (Torrence, Calif.), and PerSeptive BioSystems (Framingham, Mass.).

### Membrane Cartridge

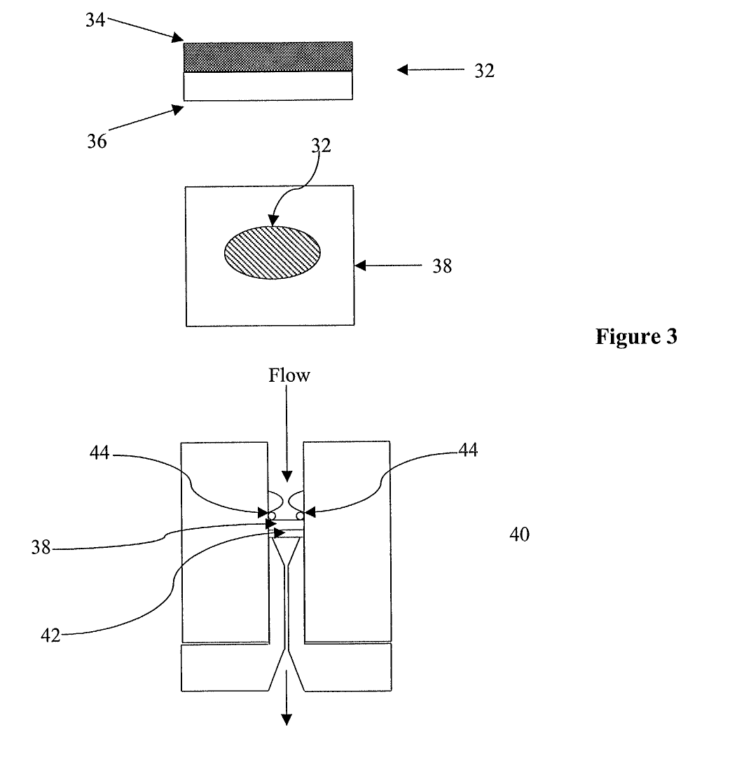

FIG. 3 provides a schematic of a membrane cartridge of the invention, which is designed to hold a membrane and to withstand high pressure. Suitable membranes have a pore size of a sufficient diameter such that proteins can pass through, but cells and large cellular components such as mitochondria, nuclei, and cellular membranes are retained by the membrane. Thus, the membrane cartridge acts as a crude filter and removes undesired material from a biological sample (e.g., blood sample) that has been applied to the membrane. Generally, membranes having pores of about 80 to about 200 μM in diameter are useful. In particular, membrane 32, which is shown in FIG. 3, is useful and is composed of absorbent pad 34, which prevents sample runoff, and membrane filter 36, which allows proteins to pass through. For example, a Stratapore™ bonded membrane (Millipore, Bedford, Mass.) containing an absorbent pad bonded to a membrane with a pore size of about 120 to about 140 μM is particularly useful. Absorbent pad 34 further can include anticoagulant agents such as heparin or EDTA to prevent blood clotting. An alternative to a bonded membrane includes the placement of filter paper in contact with the membrane such that a sample that is applied to the filter paper is absorbed and is able to traverse the membrane. Membranes and absorbent pads or filter papers that are useful have low protein binding properties (≲80 μg/cm²) and are hydrophilic.

Membrane 32 can be placed in card 38, such as that shown in FIG. 3, and placed in membrane cartridge 40. Membrane cartridge 40 is fabricated from a material such as a metal (e.g., stainless steel), metalloid, glass, ceramic, graphite, organic polymer, (e.g. PEEK) or a composite of such materials. Stainless steel is particularly useful. Membrane cartridge 40 is commercially available from UpChurch Scientific (Oak Harbor, Wash.). Card 38 is placed in membrane cartridge 40 such that liquid can flow across absorbent pad 34 into membrane filter 36, and then into membrane support 42 (e.g, a frit). Sealing device 44 (e.g., o-ring or tolerance fitting) is used to prevent leakage around membrane 32.

Membrane cartridges also include affinity membrane cartridges. Such affinity membrane cartridges can be configured as for a preconcentrator cartridge, with the exception that the membrane is derivatized with an affinity agent (e.g., antibody). Suitable membranes are composed of a chemically inert organic polymer matrix, including PTFE or TEFLON™.

### Mass Spectrometers

As used herein, a mass spectrometer can be any spray type of mass spectrometer. For example, ion spray, sonic spray, or electrospray ionization mass spectrometers can be used. Electrospray ionization mass spectrometers include microspray and nanospray type mass spectrometers. In general, electrospray mass spectrometry involves formation of ions from analytes by applying a high voltage to the sample, separating the ions according to their mass-to-charge ratio, and subsequently using a detector to generate a mass spectrum obtained from the separated ions as a result of their having passed through an electric field. Flow rate typically is about 0.001 to 0.01 ml per minute. Mass spectrometry offers ±5 dalton mass resolution of analytes. Molecular weight of analytes will vary by 0.0001% to about 0.1%, depending on the type of mass spectrometer and conditions that are used. Thus, when a peak of a mass spectrum is given a particular designation herein, it should be appreciated that the molecular weight may differ by 0.0001% to about 0.1% if a different type of mass spectrometer is used or if conditions are modified. For example, a peak designated “78,399 D” may vary by up to 78 D.

### Methods for Detecting Analytes

Systems of the invention allow the presence or absence of an analyte to be detected rapidly from a biological sample in an on-line fashion. As used herein, “on-line” refers to a system physically or electrically connected such that a biological sample can be processed in a continuous manner. As used herein, “biological sample” refers to samples containing cells or cellular material. For example, suitable biological samples include blood, plasma, urine, saliva, sputum, tears, amniotic fluid, vitreous humor, and cerebrospinal fluid. As used herein, detecting the presence or absence of an analyte includes qualitative and quantitative measurements. Thus, a sample may be classified as lacking the analyte, as containing the analyte, and, in samples classified as containing the analyte, the amount of analyte that is present can be detected.

In general, biological macromolecules such as nucleic acids, lipids, or polypeptides, are selectively eluted from a membrane cartridge, e.g., membrane cartridge 12 or affinity membrane cartridge 16, that includes a membrane, to which a biological sample has been applied. The biological sample, such as blood, can be applied to the membrane by a patient. For example, a sample collection kit containing the membrane, a needle stick, and a cartridge holder can be sent to the patient such that after finger-pricking, a drop of blood can be applied to the membrane by the patient. The kit further can include a capillary tube for applying a determined volume of blood. Alternatively, a blood collection kit can be sent to a patient such that the patient can obtain a blood sample by finger-pricking and return the blood sample to a laboratory in packaging provided in the blood collection kit. Such blood samples can be applied to the membrane by laboratory personnel or a robot.

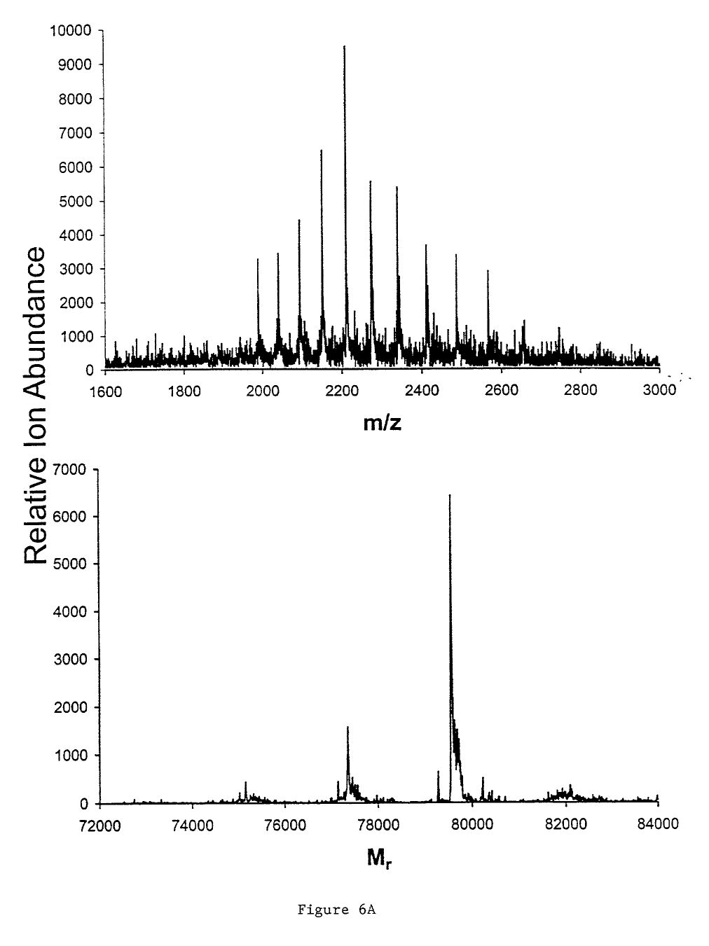

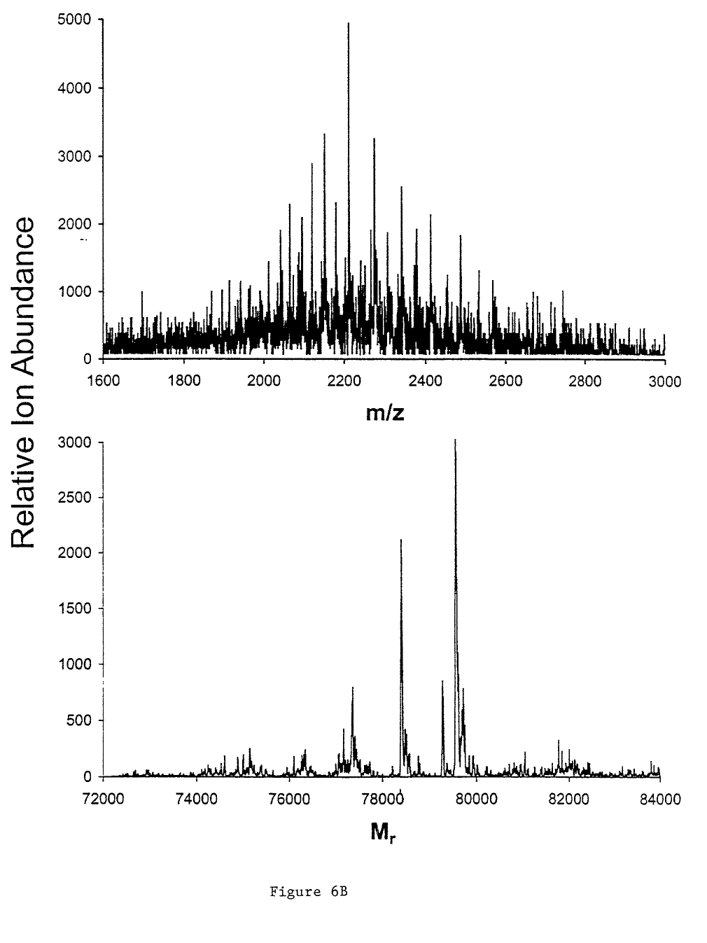

The membrane of membrane cartridge 2 or affinity membrane cartridge 16 further can include an internal standard, which is useful for measuring the amount of analyte present in the biological sample. For example, when measuring CDTs, neuraminidase treated transferrin can be used as an internal standard, and can be applied to a membrane of affinity membrane cartridge 16, which is coated with antibodies having specific binding affinities for transferrin. In particular, after neuraminidase treatment, the fraction of the digest yielding a peak designated 78399.2D on a mass spectrum (using an electrospray ionization mass spectrometer) is isolated and used as an internal standard (see, for example, FIG. 6B). The temporal progression of transferrin's molecular weight reduction from approximately 79,560D to 78,399D results from the removal of 4 sialic acid residues from apotransferrin. The resultant desialylated transferrin has a molecular weight that is clearly higher than that found for CDGS transferrin and/or for the species elevated in transferrin from alcoholics' serum.

In practice, a known amount of such an internal standard can be spiked into the biological sample obtained from the patient or placed directly onto a membrane. The amount of the internal standard (e.g., 78,399 D peak) then can be measured and compared with the size of a peak designated “77355 D”, which appears in CDGS patients and alcoholics and represents transferrin containing a single oligosaccharide moiety. A patient is diagnosed as having CDGS or as being an alcoholic if the peak designated “77355 D” appears in a range exceeding that found in control samples.

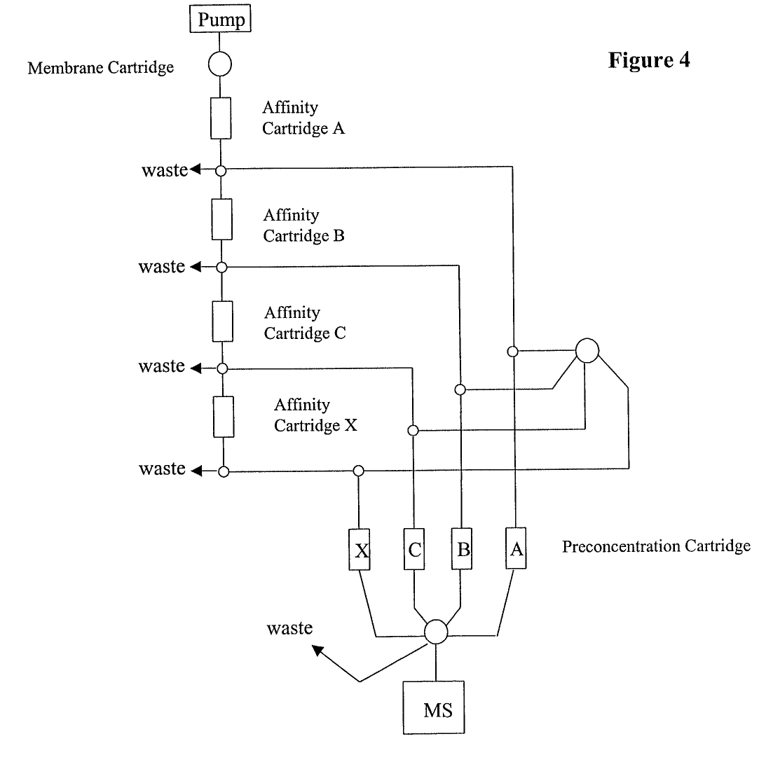

Typically, the membrane containing the drop of blood is hydrated prior to the analysis. For example, the membrane can be hydrated by addition of solvent, such as water, buffer, or an organic solvent. After hydration, analytes can be eluted from the membrane using an appropriate solvent. For example, for elution of a blood component such as transferrin, an aqueous buffer containing 150 mM NaCl, 10 mM phosphate pH 7.4 can be used. To analyze multiple analytes from a biological sample such as blood, analytes eluted from the membrane are pumped through a series of affinity cartridges, with each analyte eluted from an affinity cartridge pumped to a designated preconcentration cartridge. FIG. 4 provides a schematic representation of such a system.

The invention will be further described in the following examples, which do not limit the scope of the invention described in the claims.

### EXAMPLES

### Example 1 - Methods

Rabbit antitransferrin coupled to sepharose was prepared by mixing antibody (Dako Corp., Carpinteria, Calif.) with CNBr-activated Sepharose 4B (Pharmacia Biotech Inc., Piscataway, N.J.) under standard coupling conditions. Antitransferrin resin was stored suspended in 1:1 (v/v) 0.1 M citrate, 0.025 M phosphate, pH 7.2. Transferrin was isolated from patients' serum by rotating 1 ml of serum with 2 ml of antibody sepharose suspension for 2 hours at room temperature. After binding, the resin and liquid were transferred to a 4 ml syringe/column fitted with a frit (Alltech, Deerfield, Ill.). The binding mixture was expelled and unbound proteins were washed away in 16×1 ml washes. Transferrin was eluted with 4×1 ml washes of elution buffer (citrate/phosphate 0.1 ml/0.025 ml, pH 2.9), and immediately neutralized with Na₂HPO₄ to a pH of 7.2. After overnight dialysis of the neutralized eluate vs. 4 L 0.5 mol/l Na₂PO₄, the transferrin was iron saturated by addition of 50 μl of 20 mmol/L ferric citrate and incubated for 1-hour at 37° C. The transferrin was concentrated in an Amicon Centricon-30 microconcentrator and protein concentration was determined by the Lowry method.

Sialic acid moieties of apotransferrin (Sigma Chemical Co., St. Louis, Mo.) were cleaved to varying extents by treatment with Vibrio cholerae neuraminidase (Sigma Chemical Co., St. Louis, Mo.) according to the method of Scahuer, In: Ginsburg, V. ed., Methods in Enzymology, N.Y., Academic Press, 1978, 64-89. After exposure of 10 mg apotransferrin in 10 ml acetate/CaCl₂ buffer from 0 to 24-hours, 100 μl samples were neutralized with 900 μl cold phosphate buffered saline. Transferrin was re-isolated, iron saturated, and concentrated using methods identical to those for isolation of transferrin from serum.

Serum transferrin preparations were diluted to between 0.5 and 1.0 μg/μl protein, depending on their concentration, and 15 μl were applied to the focusing gels or used for mass spectroscopy as described below. Neutralized neuraminidase-treated transferrin was handled identically except protein concentration was not predetermined. Isoelectric focusing (IEF) gels were prepared with 6% acrylamide and a pH gradient from pH 3.0 to 10.0. The gels were focused until 1940 volt hours were reached at 5 watts (c.a. 2 hrs.). The gels were fixed with 11.5% trichloroacetic acid (TCA)/3.5% 5-sulfosalicylic acid for 30 minutes, rinsed with double distilled H₂O (ddH₂O), and then soaked in ddH₂O for 1 hour. Transferrin bands were visualized by staining with 0.5% Coomassie Brilliant Blue in 25% ethanol and 10% acetic acid and destaining for 1 hours with 40% methanol, 10% acetic acid. Gels were preserved for photography and densitometry by soaking in 2.5% glycerol for 15 minutes. Scanning was accomplished using the Helena Rep densitometer.

Automated analyses for transferrins were preformed on a Sciex API365 mass spectrometer using electrospray ionization. Since the transferrins were present in a phosphate buffer, a reverse phase guard cartridge was used as a desalting and buffer exchange step before introduction into the mass spectrometer. A Shimadzu LC system, consisting of dual LC-10AD pumps and a SCL-10A VP controller, was used in conjunction with a Perkin-Elmer Series 200 autosampler. LC and autosampler operations were controlled through the mass spectrometer data system.

A reverse phase guard cartridge (1 by 10 mm, PLRP-S, 4000 Angstrom pore size, Michrom BioResources Inc., Auburn, Calif.) was installed as the sample loop of the LC autosampler. Total volume of the cartridge plus connecting tubing was approximately 10 μl. The outlet port of the autosampler valve was connected directly to the mass spectrometer by a 50 cm length of either fused silica tubing (360 μm i.d. by 50 μm i.d.) or PEEK tubing (1/16 inch i.d. by 0.003 inch i.d.). Since sample was preconcentrated on the guard column, injection volumes many times larger than the sample loop volume can be used, while non-retained buffer components pass through the guard cartridge.

After injection of the sample, an LC gradient was developed starting from 95% A and 5% B, which was held for 3 minutes following injection, then changed to 20% A and 80% B over the next 5 minutes. After 5 minutes at 80% B and 20% A, the system was re-equilibrated to 5% B and 95% A. Mobile phase A consisted of water/acetonitrile/n-propanol/acetic acid/trifluroacetic acid in the ratio of 98/1/1/0.5/0.02 by volume. Mobile phase B was acetonitrile/n-propanol/water/acetic acid/trifluroacetic acid with a volume ratio of 80/10/10/0.5/0.02. The mobile phase flow rate was 50 μl/min. A splitting tee on the Sciex ESI interface was used to reduce the flow into the mass spectrometer to 10-20 μl/min. Under these conditions, transferrin eluted into the mass spectrometer between 6 and 10 minutes.

Spectra acquired during the elution of the transferrins were summed. The envelope of multiply charged ions was transformed to molecular mass using the BioSpec Reconstruct module of the Sciex BioMultiView software (Version 1.3b1D). Multiply charged spectra were transformed through 5 iterations, using input data between m/z 1600 and 3000, and an output data range of mass 72000 to 84000.

### Example 2-Detection of CDT by Mass Spectrometry

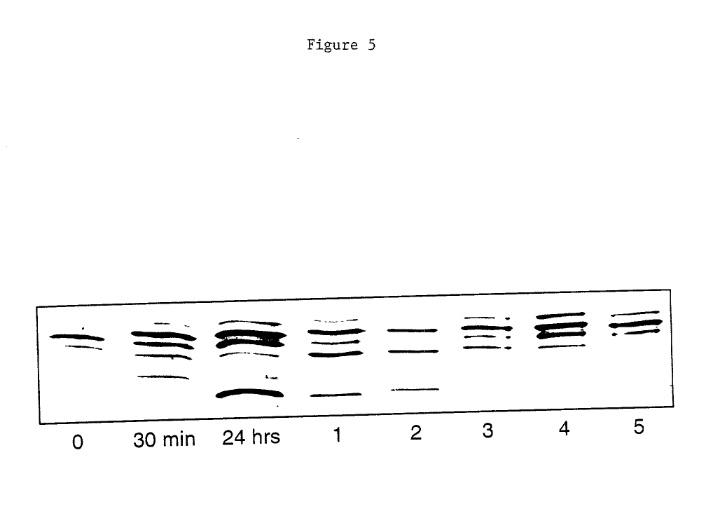

Neuraminidase digestion causes sequential detectable shifts of transferrin which focus at higher PI's, similar to the asialo, monosialo, and disialo transferrins (FIG. 5, lanes labeled 0, 30 mm and 24 hrs.). IEF shifts seen in the transferrin samples result in a transferrin pI distribution that is qualitatively very similar to the pattern seen in CDGS patients (FIG. 5, lanes 1-3) or in a presumed chronic alcoholic patient (FIG. 5, lane 4) when CDT is observable. On this basis, it would not be possible to predict the nature of the oligosaccharide structure in CDGS or alcoholism beyond suggesting that sialic acid residues are missing.

Simultaneous with the shift to higher PI caused by neuraminidase treatment, the mass spectra shows a stepwise appearance of transferrins reduced by approximately 1, 3, and 4 sialic residues (FIGS. 6A and 6B). The species appear as mass reductions in approximate multiples of 291 which is essentially identical to the calculated 292 mass difference resulting from loss of single N-acetyl-neuraminidase acid residue. This provides evidence that CDT in alcoholism result from more substantial saccharide deletion than merely loss of sialic acid.

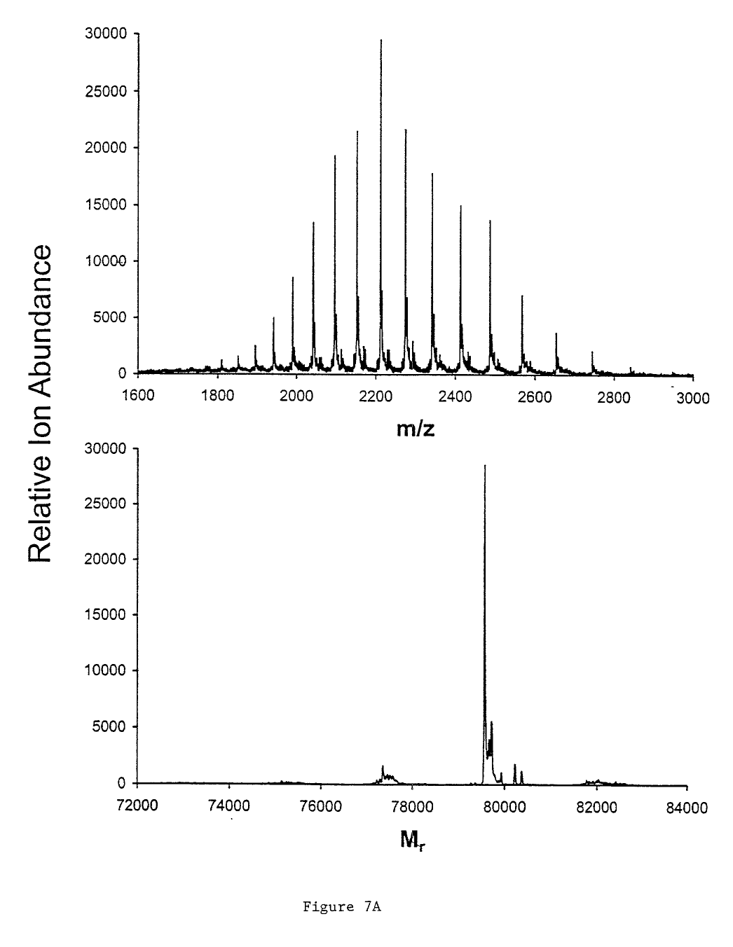

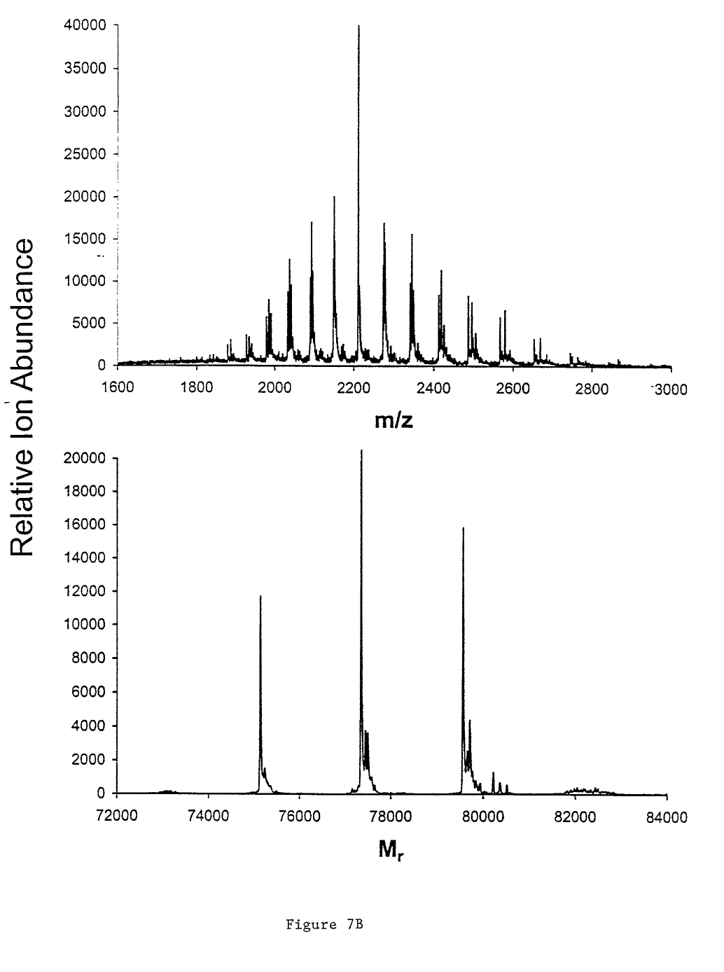

FIGS. 7A-7B are mass spectra of control samples from a normal patient (7A) and a CDGS patient (7B). The transferrin isolated from serum of patients with CDGS have the standard 79,560 D peak and a major peak at 77,350 D, consistent with loss of slightly more than 2,200 daltons or complete oligosaccharide moieties. This is seen in CDGS patients with both phosphomannomutase deficiency and phosphomannoisomerase deficiency and implies the resultant deficiency of GDP-mannose causes loss of transfer of immature oligosaccharides at the dolichol glycosylation step. When the second glycosylation site is also vacant, a third major species at 75,140 appears. This is accounted for by the asialo band in IEF. Transferrin lacking both oligosaccharide chains was apparent in most CDGS sera. Relative amounts of the two under- glycosylated transferrins occur to variable degrees, both between patients and temporally within individual patients.

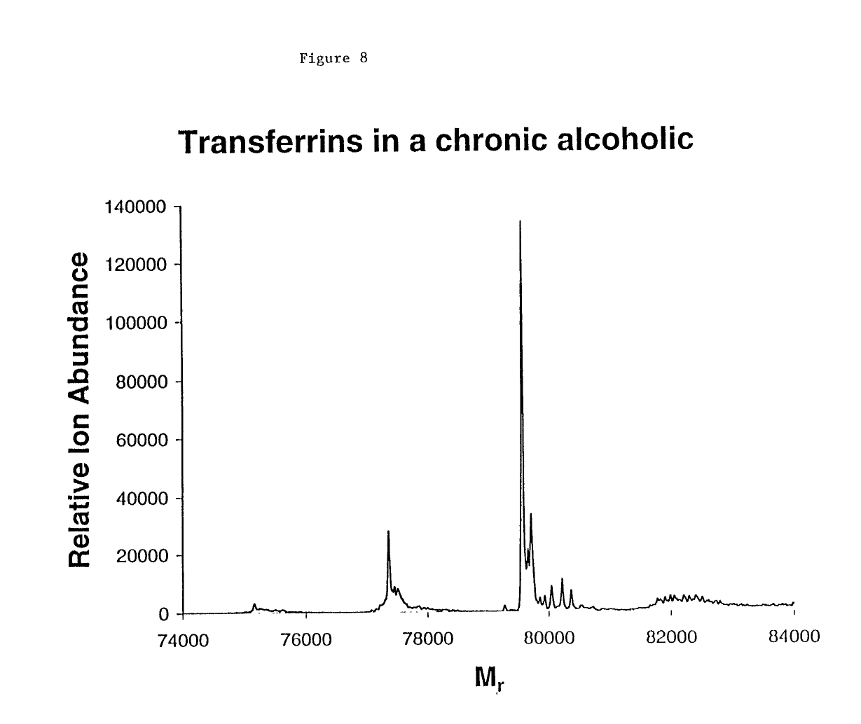

A mass spectrum of transferrin isolated from the serum of a chronic alcoholic is shown in FIGS. 8. As would be expected from the subtle difference in the IEF pattern when compared with control serum, the appearance of an abnormal mass peak, though reproducible, is far less dramatic than found in CDGS patients. The qualitative observation that can be made is that CDT arises at the point at or before oligosaccharide transfer in the rough endoplasmic reticulum similar to the site affected in CDGS.

### Example 3-On-Line Measurement of CDT

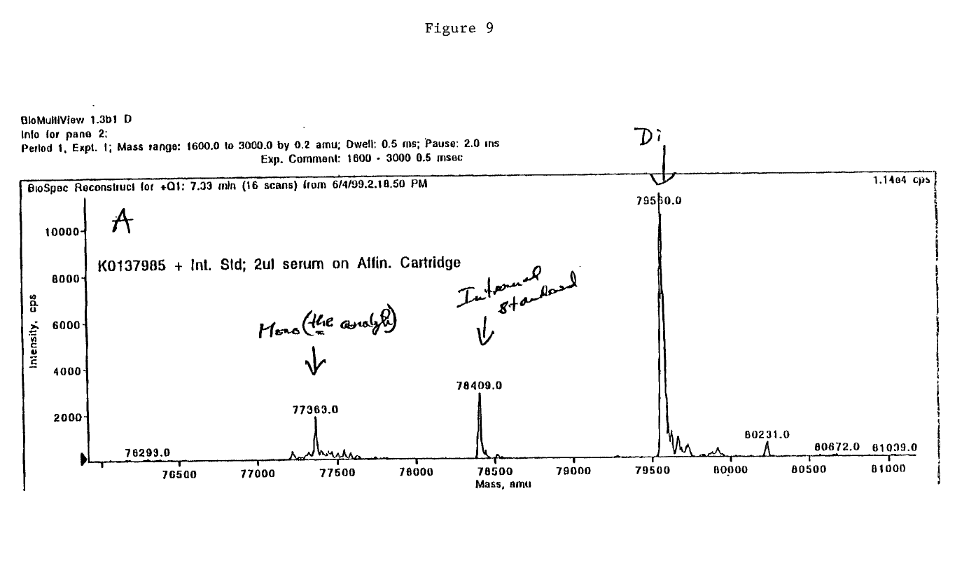

An internal standard was prepared by digesting apotransferrin with Vibrio cholerae neuraminidase as described in Example 1. The species of transferrin having a mass of 78399±10 D was purified. A known amount of the internal standard (2 μg) was spiked into 2 μl of a serum sample from an alcoholic. The serum/internal standard mixture then was applied to an affinity cartridge (see affinity cartridge 2 of FIG. 1) and processed on-line through preconcentration cartridge 4 and electrospray ionization mass spectrometer 8. As indicated in FIG. 9, the internal standard is clearly distinguished from the analyte of interest.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A system for detecting an analyte, said system comprising an immunoaffinity cartridge, a preconcentrator cartridge, and a mass spectrometer, wherein said preconcentrator cartridge operably connects said immunoaffinity cartridge and said mass spectrometer, and wherein said preconcentrator cartridge comprises a container having an inner surface, an outer surface, a first port, a second port, and a membrane disposed inside said container in contact with said inner surface of said container such that a liquid sample that enters said container through said first port and exits said container through said second port traverses said membrane, wherein said membrane comprises a chemically inert organic polymer matrix embedded with absorbent particles.

2. The system of claim 1, wherein said immunoaffinity cartridge comprises an anti-transferrin antibody.

3. The system of claim 1, wherein said immunoaffinity cartridge comprises a single compartment.

4. The system of claim 1, wherein said immunoaffinity cartridge comprises a multiple compartment.

5. The system of claim 2, wherein said antibody is polyclonal.

6. The system of claim 2, wherein said antibody is monoclonal.

7. The system of claim 1, wherein said mass spectrometer is an electrospray ionization-mass spectrometer.

8. The system of claim 7, wherein said electrospray ionization-mass spectrometer is a microspray electrospray ionization-mass spectrometer or a nanospray electrospray ionization-mass spectrometer.

9. The system of claim 1, wherein said system further comprises a membrane cartridge, wherein said membrane cartridge is operably connected to said immunoaffinity cartridge.

10. A system for detecting an analyte, said system comprising a membrane cartridge, an immunoaffinity cartridge, a preconcentrator cartridge, and a mass spectrometer, wherein said membrane cartridge is operably connected to said immunoaffinity cartridge, said immunoaffinity cartridge is operably connected to said preconcentrator cartridge, and said preconcentrator cartridge is operably connected to said mass spectrometer.

11. The system of claim 10, wherein said membrane cartridge comprises a membrane.

12. The system of claim 11, wherein said membrane comprises a chemically inert organic polymer matrix of a defined pore size.

13. The system of claim 11, wherein said preconcentrator cartridge comprises a membrane.

14. The system of claim 13, wherein said membrane is a chemically inert organic polymer matrix embedded with absorbent particles.

15. The system of claim 10, wherein said membrane cartridge comprises a container having an inner surface, an outer surface, a first port, a second port, and a membrane disposed inside said container in contact with said inner surface of said container such that a liquid sample that enters said container through said first port and exits said container through said second port traverses said membrane.

16. The system of claim 10, wherein said preconcentrator cartridge comprises a container having an inner surface, an outer surface, a first port, a second port, and a membrane disposed inside said container in contact with said inner surface of said container such that a liquid sample that enters said container through said first port and exits said container through said second port traverses said membrane.

17. The system of claim 10, wherein said mass spectrometer is an electrospray ionization-mass spectrometer.

18. The system of claim 17, wherein said electrospray ionization-mass spectrometer is a microspray electrospray ionization-mass spectrometer or a nanospray electrospray ionization-mass spectrometer.

19. The system of claim 10, wherein said immunoaffinity cartridge comprises an anti-transferrin antibody.

20. The system of claim 19, wherein said antibody is polyclonal.

21. The system of claim 19, wherein said antibody is monoclonal.

22. A method for detecting the presence or absence of an analyte in a biological sample, said method comprising:
(a) eluting biological macromolecules from a membrane cartridge comprising a membrane, wherein said membrane comprises said biological sample;
(b) immunopurifying said analyte from said eluted biological macromolecules;
(c) concentrating said immunopurified analyte; and
(d) detecting the presence or absence of said concentrated analyte by mass spectrometry, wherein steps (a)-(d) are performed on-line.

23. The method of claim 22, wherein said biological sample is a blood sample.

24. The method of claim 22, wherein said biological macromolecules and said analyte each comprise polypeptides.

25. The method of claim 24, wherein said analyte is carbohydrate-deficient transferrin.

26. The method of claim 22, wherein said membrane cartridge comprises a container having an inner surface, an outer surface, a first port, a second port, and said membrane disposed inside the container in contact with the inner surface of the container such that a liquid sample that enters the container through said first port and exits said container through said second port traverses said membrane.

27. The method of claim 22, wherein said membrane comprises a chemically inert organic polymer matrix of a defined pore size.

28. The method of claim 27, wherein said membrane further comprises an internal standard.

29. The method of claim 28, wherein said internal standard is transferrin lacking sialic acid residues.

30. A method of evaluating a patient for the presence or absence of an analyte in a biological sample from said patient, said method comprising:
(a) providing a sample collection kit to said patient, said kit comprising a membrane and instructions for obtaining said biological sample and applying said biological sample to said membrane to form a sample membrane;
(b) receiving said sample membrane from said patient;
(c) inserting said sample membrane into a membrane cartridge;
(d) eluting biological macromolecules from said membrane cartridge;
(e) immunopurifying said analyte from said eluted biological macromolecules;
(f) concentrating said immunopurified analyte; and
(g) detecting the presence or absence of said concentrated analyte by mass spectrometry, wherein steps (d)-(g) are performed on-line.
